(19) United States
(12) Patent Application Publication
(10) Pub. No.: US 2011/0135742 A1
(43) Pub. Date: Jun. 9, 2011

(54) CONTROLLED RELEASE ENCAPSULATED ANTI-BACTERIAL AND ANTI-INFLAMMATORY NANOPARTICLES

(75) Inventors: Jenny Kim, Pacific Palisades, CA (US); Adam Friedman, New York, NY (US); Robert Modlin, Sherman Oaks, CA (US)

(73) Assignee: The Regents of the University of California, Oakland, CA (US)

(21) Appl. No.: 12/303,166

(22) PCT Filed: Jun. 19, 2007
(86) PCT No.: PCT/US07/71580

§ 371 Date: Jan. 19, 2011

Related U.S. Application Data
(60) Provisional application No. 60/815,286, filed on Jun. 20, 2006.

(51) Int. Cl. A61K 31/722; A61K 9/14; A61K 38/39; A61K 31/7056; A61K 33/00; A61K 31/7036; A61K 38/16; A61K 31/122; A61P 31/00; A61P 17/00; A61P 29/00; B82Y 5/00

(52) U.S. Cl. 424/499; 514/55; 514/17.2; 514/24; 424/717; 514/40; 514/18.6; 424/94.1; 977/773; 977/915

(57) ABSTRACT

This invention pertains to the formulation of nanoparticles that have intrinsic antimicrobial and anti-inflammatory activity. The nanoparticles can be impregnated with one or more therapeutic agents and thereby enhance the antimicrobial and/or anti-inflammatory activity of such agents, and also other properties that the therapeutic agents provide.

### CROSS-REFERENCE TO RELATED APPLICATIONS

This application claims benefit of and priority to U.S. Ser. No. 60/815,286, filed Jun. 20, 2006, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. AI59091 and AR48551, awarded by the National Institutes of Health. The Government of the United States of America has certain rights in this invention.

### FIELD OF THE INVENTION

This invention pertains to antimicrobial and anti-inflammatory therapeutic agents. In particular, nanoparticles having intrinsic anti-inflammatory and/or antimicrobial activity are provided.

### BACKGROUND OF THE INVENTION

The benefits from the use of antibiotics as a means of treating infections have been increasingly compromised by the development of resistant strains of microorganisms. One approach taken by manufacturers of antimicrobial agents is to devise agents to which the organisms are not resistant. Because considerable time and resources have been spent on the development of current antimicrobial agents, it is desirable to develop antimicrobial enhancer agents that, when administered with an antimicrobial agent, render the otherwise resistant microbes sensitive to the antimicrobial agent.

Antimicrobial agents have proved of particular value in the treatment of pathologies characterized by infections or inflammation of the skin. One such pathology is acne.

Acne is probably the most renowned lay dermatology condition in modern society. Acne can range from existing as a slight annoyance or even an embarrassing condition, to painful, scarring, inflammatory lesions. Regardless of age, the four key pathogenetic factors of acne, for the most part, remain constant. These include follicular epithelial hyperproliferation with follicular plugging, excess sebum production, inflammation, and the colonization of Propionibacterium. Acne vulgaris begins with the microcomedo lesion. Though some contest that acne is created by follicular plugging, rather then inflammation or bacterial colonization, others have demonstrated that inflammation is the primary process that results in abnormal hypercornification. Another hypothesis suggests that androgen hormones are to blame, shifting follicular linoleic acid levels, and interleukin-1α. The microcomedone can eventually become loaded with P. acnes, whose byproducts are chemoattractants as well as proinflammatory stimulants. Prompting of inflammatory cells results in enzymatic disruption of the follicular wall. The range of inflammatory involvement varies, from immune sensitivity to P. acnes as well as gland response to circulating androgens.

Today, an entire arsenal of acne therapeutics is readily available. Benzoyl peroxide has been used as an over the counter therapeutic to treat acne for decades. It is known to be antimicrobial, anti-inflammatory, as well as comedolytic. It is a good treatment for mild to moderate acne, especially when used in combination with salicylic acid and sulfa. Fortunately, bacterial resistance to benzoyl peroxide has not been reported. Yet, many users complain of dry or scaling skin following use, and therefore seek out alternatives.

Today, retinoids have become the mainstream treatment for moderate to severe acne. Topical retinoids, such as Tazarotene, or Tazarac, have become well accepted as the treatment of choice for comedonal acne. Furthermore, they are also considered effective against inflammatory acne. Topical retinoids can be used alone as well as in conjunction with other treatment. However, skin irritation is a common side effect, limiting their application due to patient resistance and noncompliance.

Isotretinoin (typically marketed (ACCUTANE® (Roche), AMNESTEEM® (Mylan), CLARAVIS® (Barr), SOTRET® (Ranbaxy), or ROACCUTANE® (Roche)), is easily the most effective and well known acne medication on the market. It is recorded as being 80-90% successful. However, the numerous potential side effects limit its use for those who truly require it, for example, those patients who do not respond to first line topical and systemic acne treatments. The elevation of triglycerides, the teratogenic effects, and the potential depression or psychosis which follow use have all brought much controversy to the utilization of this medication, so much so that it has prompted the development of a unified registry through which one must go through in order to prescribe the treatment.

Antibiotics also play an essential role in the treatment and management of acne. Doxycycline and minocycline have replaced tetracycline as first-line anticomedomal antibiotics due to building resistance and its numerous side effects. There is actually a great deal of concern over prolonged use of antibiotics because of the risk of emerging resistant species as well as colonization with potentially life threatening pathogens. Though P. acnes resistance to both tetracycline and erythromycin is a widely accepted fact, what truly worries physicians is the development of resistance to dangerous microbes such as Streptococcus pyogenes. One study found that eighty-five percent of S. pyogenes cultured from individuals on chronic antibiotics for acne management was resistant to at least one form of tetracycline.

In view of the foregoing, it will be recognized that: 1) Much is known regarding the pathogenesis of Acne vulgaris; 2) Varying treatments have been developed over the last three decades and are continuing to be studied; and 3) Good therapeutics typically come at the cost of side effects.

### SUMMARY OF THE INVENTION

In certain embodiments this invention pertains to the use of nanoparticles as a delivery modality for drugs, particularly for drugs used in the treatment of skin diseases. Nanoparticles have thus far demonstrated excellent medicinal controlled delivery and stabilization. Using the actual nanoparticles, which are nontoxic, biodegradable, and biocompatible, as the actual therapeutic is ideal. We demonstrated that the nanoparticles of the present invention can serve both as a vehicle for delivery, but even more importantly, alone as an efficacious antibacterial and/or anti-inflammatory, and/or anti-viral, and/or anti-fungal preparation.

Nanoparticles are particularly advantageous in the formulations of the present invention. The have a long shelf life and can encapsulate a great range of therapeutics. Moreover it was a surprising discovery that the nanoparticles themselves have anti-microbial and/or anti-inflammatory activity. We show that CFU assays of P. acnes, S. aureus, and E. coli treated with these nanoparticles demonstrated effective killing impact with increasing concentrations of nanoparticles dispersed in distilled water, suggesting that the nanoparticles of the present invention alone, and/or impregnated with one or more therapeutic agents, and/or in conjunction with one or more therapeutic agents can be used as an effective anti-microbial and/or anti-inflammatory therapeutic. It has also been demonstrated that the number of nanoparticles that can cross mucosal membranes is significantly greater than that of microspheres.

Accordingly, in certain embodiments, this invention provides an antimicrobial composition. The composition typically comprises particles comprising chitosan and alginate where the particles have antimicrobial activity. In various embodiments the particles are nanoparticles (e.g., having an average diameter of less than about 500 nm, less than about 250 nm, or less than about 150 nm or 100 nm). In certain embodiments the particles have antimicrobial activity against bacteria selected from the group consisting of S. aureus, B. circulans, B. cereus, E. coli, P. vulgaris, P. acnes, S. enterica, V. anguillarum, K. pneumoniae, P. piscicida, P. aeruginosa, A. tumefaciens, C. micgiganence, A. mali, E. chrysanthemi, X. campestris, C. diplodiella, and P. piricola. In various embodiments the chitosan is partially or fully deacetylated. In various embodiments the chitosan is at least 50% deacetylated, or at least 80% deacetylated, or at least 85% deacetylated, or at least 90% deacetylated. In various embodiments the particles (nanoparticles) are impregnated with one ore more biologically active agent(s). In certain embodiments the biologically active agent(s) comprise an agent for the treatment of a skin disorder. In various embodiments the biologically active agent is selected from the group consisting of sodium bicarbonate, benzoyl peroxide, retinoids, retin A, Clindamycin, Tetracycline, flagyl, psuedomonic acid, gentimycin, Isotretinoin, Azelaic acid, Adapalene, sodium sulfacetamide, salicylic acid, sulfur, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and steroids. In various embodiments the biologically active agent is selected from the group consisting of diprolene, lidex, triamcinolone, elocon, and westcort.

In various embodiments methods are provided for mitigating one or more symptoms of a skin pathology characterized by a microbial infection and/or inflammation. The methods typically involve contacting the infected skin with a composition comprising particles comprising chitosan and alginate where the particles have antimicrobial activity. In various embodiments the particles are nanoparticles (e.g., having an average diameter of less than about 500 nm, less than about 250 nm, or less than about 150 nm or 100 nm). In certain embodiments the particles have antimicrobial activity against bacteria selected from the group consisting of S. aureus, B. circulans, B. cereus, E. coli, P. vulgaris, P. acnes, S. enterica, V. anguillarum, K. pneumoniae, P. piscicida, P. aeruginosa, A. tumefaciens, C. micgiganence, A. mali, E. chrysanthemi, X. campestris, C. diplodiella, and P. piricola. In various embodiments the chitosan is partially or fully deacetylated. In various embodiments the chitosan is at least 50% deacetylated, or at least 80% deacetylated, or at least 85% deacetylated, or at least 90% deacetylated. In various embodiments the particles (nanoparticles) are impregnated with one ore more biologically active agent(s). In certain embodiments the biologically active agent(s) comprise an agent for the treatment of a skin disorder. In various embodiments the biologically active agent is selected from the group consisting of sodium bicarbonate, benzoyl peroxide, retinoids, retin A, Clindamycin, Tetracycline, flagyl, psuedomonic acid, gentimycin, Isotretinoin, Azelaic acid, Adapalene, sodium sulfacetamide, salicylic acid, sulfur, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and steroids. In various embodiments the biologically active agent is selected from the group consisting of diprolene, lidex, triamcinolone, elocon, and westcort.

Methods are also provided for reducing the inflammation of a tissue. The methods typically comprise contacting the tissue with a composition comprising particles comprising chitosan and alginate where the particles have anti-inflammatory activity. In various embodiments the particles are nanoparticles (e.g., having an average diameter of less than about 500 nm, less than about 250 nm, or less than about 150 nm or 100 nm). In certain embodiments the particles have antimicrobial activity against bacteria selected from the group consisting of S. aureus, B. circulans, B. cereus, E. coli, P. vulgaris, P. acnes, S. enterica, V. anguillarum, K. pneumoniae, P. piscicida, P. aeruginosa, A. tumefaciens, C. micgiganence, A. mali, E. chrysanthemi, X. campestris, C. diplodiella, and P. piricola. In various embodiments the chitosan is partially or fully deacetylated. In various embodiments the chitosan is at least 50% deacetylated, or at least 80% deacetylated, or at least 85% deacetylated, or at least 90% deacetylated. In various embodiments the particles (nanoparticles) are impregnated with one ore more biologically active agent(s). In certain embodiments the biologically active agent(s) comprise an agent for the treatment of a skin disorder. In various embodiments the biologically active agent is selected from the group consisting of sodium bicarbonate, benzoyl peroxide, retinoids, retin A, Clindamycin, Tetracycline, flagyl, psuedomonic acid, gentimycin, Isotretinoin, Azelaic acid, Adapalene, sodium sulfacetamide, salicylic acid, sulfur, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and steroids. In various embodiments the biologically active agent is selected from the group consisting of diprolene, lidex, triamcinolone, elocon, and westcort.

In various embodiments methods are provided for increasing the antimicrobial and/or anti-inflammatory activity of a biologically active agent. The methods typically involve combining the agent with particles comprising chitosan and alginate where the particles have antimicrobial activity. In various embodiments the particles are nanoparticles (e.g., having an average diameter of less than about 500 nm, less than about 250 nm, or less than about 150 nm or 100 nm). In certain embodiments the particles have antimicrobial activity against bacteria selected from the group consisting of S. aureus, B. circulans, B. cereus, E. coli, P. vulgaris, P. acnes, S. enterica, V. anguillarum, K. pneumoniae, P. piscicida, P. aeruginosa, A. tumefaciens, C. micgiganence, A. mali, E. chrysanthemi, X. campestris, C. diplodiella, and P. piricola. In various embodiments the chitosan is partially or fully deacetylated. In various embodiments the chitosan is at least 50% deacetylated, or at least 80% deacetylated, or at least 85% deacetylated, or at least 90% deacetylated. In certain embodiments the biologically active agent is selected from the group consisting of where the biologically active agent is selected from the group consisting of benzoyl peroxide, retin A, Clindamycin, Tetracycline, Isotretinoin, Azelaic acid, Adapalene, sodium Sulfacetamide, salicylic acid, sulfur, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and and steroids.

In various embodiments a facial scrub is provided. The facial scrub typically comprises a cleaning agent; and antimicrobial particles comprising chitosan and alginate. In various embodiments the cleaning agent comprises a surfactant (e.g., a non-ionic, anionic, cationic, amphoteric, etc.). In various embodiments the scrub further comprises an abrasive particle. In various embodiments the particles are nanoparticles (e.g., having an average diameter of less than about 500 nm, less than about 250 nm, or less than about 150 nm or 100 nm). In certain embodiments the particles have antimicrobial activity against bacteria selected from the group consisting of S. aureus, B. circulans, B. cereus, E. coli, P. vulgaris, P. acnes, S. enterica, V. anguillarum, K. pneumoniae, P. piscicida, P. aeruginosa, A. tumefaciens, C. micgiganence, A. mali, E. chrysanthemi, X. campestris, C. diplodiella, and P. piricola. In certain embodiments the antimcriobial particles are impregnated with a biologically active agent (e.g., an agent for the treatment of a skin disorder). In certain embodiments the biologically active agent is selected from the group consisting of benzoyl peroxide, retin A, topical Clindamycin, topical Tetracycline, topical Isotretinoin, Azelaic acid, Adapalene, sodium Sulfacetamide, salicylic acid, sulfur, resorcinol, vitamin A, vitamin D, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and steroids.

Also provided is an anti-microbial composition. The composition comprises particles comprising chitosan and a hydrophilic biocompatible polymer, where the particles have “intrinsic” antimicrobial activity. In certain embodiments the biocompatible polymer is selected from the group consisting of polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methylmethacrylate), poly(ethylmethacrylate), poly(butylmethacrylate), poly(isobutylmethacrylate), poly(hexlmethacrylate), poly(isodecylmethacrylate), poly(laurylmethacrylate), poly(phenylmethacrylate), poly(methacrylate), poly(isopropacrylate), poly(isobutacrylate), poly(octadecacrylate), polyethylene, polypropylene poly(ethylene glycol), poly(ethylene oxide), poly(ethylene terephthalate), poly(vinyl alcohols), poly(vinyl acetate), poly vinyl chloride, polystyrene, polyhyaluronic acids, casein, gelatin, gluten, polyanhydrides, polyacrylic acid, alginate, any copolymers thereof, and any combination of any of these.

### DEFINITIONS

The term “antimicrobial activity” refers to the ability of a composition or compound to partially or fully inhibit the growth and/or proliferation of one or more species of microbe, e.g., various species of bacteria. In certain embodiments antimicrobial activity refers to the ability of a composition or compound to kill various species of microbe.

When a nanoparticles is referred to as having “intrinsic antimicrobial activity” this indicates that the nanoparticle has antimicrobial activity even in the absence of any other antimicrobial agent.

The term “nanoparticle” refers to a particle that has an average diameter (or characteristic dimension where the particle is not generally spherical) of less than about 800 nm, preferably less than about 500 nm, more preferably less than about 300 nm, still more preferably less than about 250 nm, even still more preferably less than about 200 nm, and most preferably less than about 150 nm or 100 nm. In certain embodiments, the nanoparticles range in size from about 50 to about 100 nm.

The phrase “in conjunction with”, when used in reference to the use of one or more agents, as described herein (e.g., nanoparticles and/or other therapeutic agents) indicates that the nanoparticles and/or drug-containing nanoparticles and the other therapeutic agent(s) are administered so that there is at least some chronological overlap in their physiological activity on the organism. Thus the two agents can be administered simultaneously and/or sequentially. In sequential administration there may even be some substantial delay (e.g., minutes or even hours or days) before administration of the second agent as long as the first administered agent has exerted some physiological alteration on the organism when the second administered agent is administered or becomes active in the organism.

The term “treat” when used with reference to treating, e.g. a pathology or disease refers to the mitigation and/or elimination of one or more symptoms of that pathology or disease, and/or a reduction in the rate of onset or severity of one or more symptoms of that pathology or disease, and/or the prevention of that pathology or disease.

### BRIEF DESCRIPTION OF THE DRAWINGS

FIG. 1 shows the results of a CFU assay showing that both chitosan and chitosan-alginate nanoparticles exhibited antimicrobial activity in a concentration-dependent manner, reducing the number of P. acnes.

FIG. 2 shows the results of a CFU assay illustrating the killing capacity of an encapsulated nonactive granulysin peptide 61-80 against P. acnes. Note the nonactive peptide (open diamond) alone does not influence bacterial growth or death. However when encapsulated with the nanoparticle from the prevent invention, there is a concentration-dependent antimicrobial activity seen against P. acnes (solid diamond). In addition, when active granulysin peptide 31-50 (open square) is encapsulated with the nanoparticle, there is enhanced killing against P. acnes (solid square).

FIG. 3, panel A, shows the results of a CFU assay of encapsulated chitosan/alginate nanoparticles and non-encapsulated chitosan against S. aureus. FIG. 3, panel B, shows the results of a CFU assay of encapsulated and non-encapsulated chitosan against E. coli.

FIG. 4 shows the results of a CFU a assay illustrating the enhanced killing capacity of a nanoparticle encapsulated benzoyl peroxide in comparison to un-encapsulated benzoyl peroxide against P. acnes. Note that benzoyl peroxide (open square) alone kills bacteria but the benzoyl peroxide encapsulated with the nanoparticle from the current invention (solid square) kills P. acnes more effectively.

FIG. 5 shows that nanoparticles inhibit production of IL-12p40 in a dose-dependent manner. P. acnes stimulated IL-12 protein production by primary human monocytes were inhibited by nanoparticles, benzoyl peroxide and nanoparticle encapsulated benzoyl peroxide in a dose-dependent manner. Each bar represents dilutions in multiples of 10:1, 10⁻¹, 10⁻².

FIG. 6 shows the results of an MTT assay demonstrating that nanoparticle, benzoyl peroxide and nanoparticle encapsulated benzoyl peroxide are not toxic to eukaryotic cells.

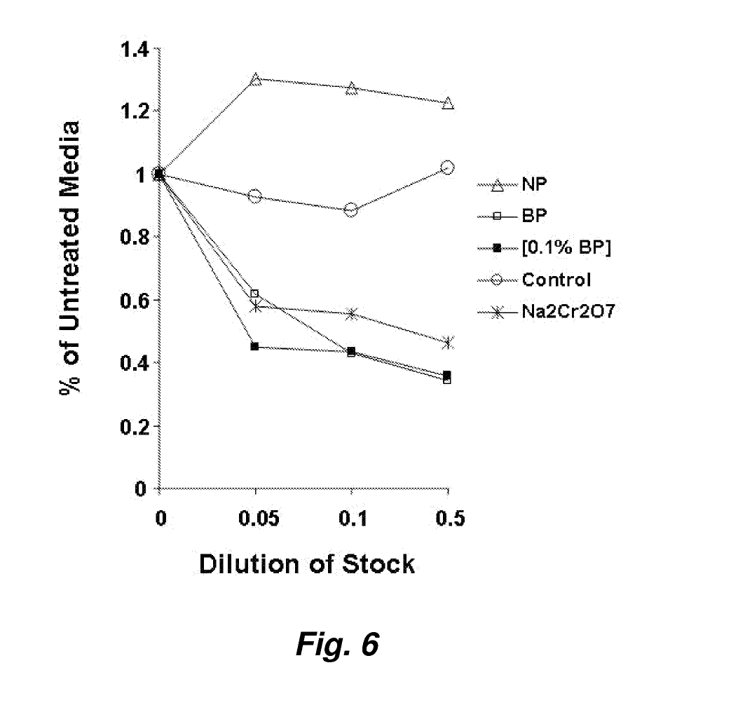

FIG. 7 shows that scanning electron microscopy demonstrates that nanoparticles (panel B), benzoyl peroxide (panel C) and encapsulated benzoyl peroxide (panel D) destroy the bacteria and kill P. acnes. The physical changes in the bacteria are shown in comparison to control (panel A).

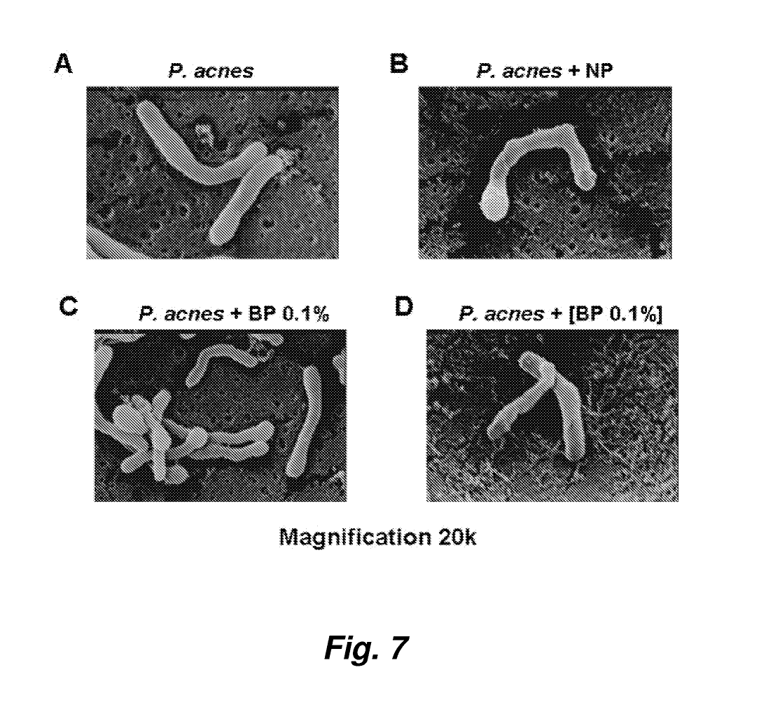

### DETAILED DESCRIPTION

This invention pertains to the development of anti-microbial (e.g., anti-bacterial) nanoparticle compositions that are useful therapeutics in their own right and that can further act as drug carriers and thereby enhance the antimicrobial activity of whatever drug(s) are carried therein.

In certain embodiments the nanoparticles comprise chitosan and a biocompatible polymer (e.g., alginate). The chitosan-based nanoparticles possess antimicrobial activity by themselves and can readily be used to carry other therapeutic agents including, but not limited to, antibiotics and anti-inflammatory drugs including but not limited to retinoids, fruit acids, antioxidants, dermal filler substances such as collagen and hyaluronic acid, peptides that are antimicrobial or enhance skin remodeling and healing, botox, and the like.

It is believed the antimicrobial nanoparticle systems of this invention offer a number of benefits. First, highly stable nanoparticles, provide maximum therapeutic benefit without risk of degradation. Second, both the size and physiochemical properties of the nanoparticles provide for increased epidermal penetrance. Third, the physiochemical properties of the nanoparticles can offer delayed release of the therapeutic activity (e.g., of the encapsulated drug(s)), providing the user with the benefit of prolonged impact long after its application. Fourth, the components of the nanoparticles possess minimal toxicity, and have already been approved by the FDA for other medicinal purposes.

Without being bound to a particular theory, it is believed that several mechanism may account for the therapeutic efficacy of the nanoparticles described herein. One mechanism is based on the interaction between positively charged chitosan molecules and negatively charged microbial cell membranes, which produces a leakage of intracellular components resulting in death of the bacteria. Another mechanism is based on the interaction of chitosan with the membrane of the cell to alter cell permeability. In addition, it is believed that chitosan can act as a chelating agent and bind trace elements, thereby inhibiting bacterial cell growth and toxin production.

It is also noted that in addition to antibacterial activity, the nanoparticles of the present invention also demonstrate anti-inflammatory activity as well as anti-fungal and anti-viral activity. Without being bound by a particular theory, it is believed that this activity may result from the ability of the nanoparticles to impede fungal adhesion and interferon induction.

As indicated above, the nanoparticles of this invention show significant antimicrobial and anti-inflammatory activity. Accordingly, the nanoparticles and nanoparticle compositions of this invention find uses in a wide variety of contexts. The nanoparticles, by themselves, or when they incorporate one or more drugs, are useful for providing topical antimicrobial activity and/or topical anti-inflammatory activity, or, in certain embodiments, the nanoparticles can be orally, nasally, rectally, or parenterally administered.

The nanoparticles and/or compositions comprising the nanoparticles can be topically administered as a simple disinfectant. Thus, for example, the nanoparticles themselves, or nanoparticles comprising one or more drugs can be used to help prevent E. coli infections. E. coli infections plague domestic and foreign populations alike, causing pathologies ranging from gastroenteritis to Hemolytic Uremic Syndrome. A preparation that quickly neutralizes the bacteria before enteric invasion is possible would spare many from these conditions.

The nanoparticles can be combined with or impregnated with one or more therapeutic agents (e.g. drugs). The nanoparticles thereby provide antimicrobial and/or anti-inflammatory activity where the therapeutic agents lack such or increase the antimicrobial and/or anti-inflammatory activity of the agents.

The nanoparticles, particular when combined with appropriate therapeutics are particularly well suited for treatment of pathologies characterized by infection and/or inflammation of the skin. Such pathologies include, but are not limited to acne and/or seborrheic dermatitis, psoriasis, contact dermatitis, atopic dermatitis, lichen planus, lichen simplex chronicus, hand dermatitis, irritant dermatitis, surgical wounds, burn wounds, chronic wounds, laser resurfacing, and infections of skin surrounding indwelling feeding tubes or ostomy sites, and the like. In certain embodiments, the nanoparticles provide an added benefit where there is secondary infection, common in atopic dermatitis. Thus, in various embodiments, the nanoparticles are combined with, used in conjunction with, or used to incorporate one or more therapeutic agents, e.g. agents used in the treatment of acne, seborrheic dermatitis, or other pathologies, e.g., as described above.

The nanoparticles when used in combination with therapeutic agents and even when used alone are also believed to be effective in the treatment of acne, impetigo, folliculitis, cutaneous staph infection, hot tub folliculitis, and the like.

In certain embodiments, the nanoparticles can also be used as a component of various cosmetic agents, e.g. soaps, cleanser, facial scrubs, creams, ointments, and the like to provide/increase the antimicrobial and/or anti-inflammatory activity of such cosmetic agents.

### I. Nanoparticle Fabrication.

In certain embodiments the anti-microbial nanoparticles of this invention comprise one or more chitosans combined with a biocompatible polymer (e.g., alginate).

A) Chitosans

Chitosans are biopolymers that belong to the group of hydrocolloids. In contrast to most hydrocolloids, which are negatively charged at biological pH values, chitosans are cationic biopolymers under these conditions. The positively charged chitosans are capable of interacting with oppositely charged surfaces and, accordingly, are typically used in cosmetic hair-care and body-care formulations and pharmaceutical formulations.

Chitosans are produced from chitin, preferably from the shell remains of crustaceans which are available in large quantities as inexpensive raw materials. Normally, the chitin is first deproteinized by addition of bases (e.g., 40% NaOH), demineralized by addition of mineral acids and, finally, deacetylated by addition of strong bases.

One process for the production of a chitin degradation product involves treating crab shells with hydrochloric acid at room temperature, then deacetylating with caustic soda solution over a period of 42 hours at 100° C., subsequently treating with more hydrochloric acid, e.g., at room temperature and, optionally, briefly after treating with sodium hydroxide solution, again at room temperature. According to this process, the deacetylation takes place in the second step. By contrast, the final treatment with sodium hydroxide solution is merely carried out to “fine tune” the degree of deacetylation and, accordingly, takes place at room temperature.

The chitosans employed in the present invention are distinguished from typical chitosans by virtue of their substantial degree of deacetylation. In various embodiments the chitosans used in the nanoparticles of this invention typically have a degree of deacetylation of greater than about 50%, 60%, or 70%, preferably a degree of deacetylation greater than about 75%, or 80%, and most preferably a degree of deacetylation of greater than about 82%, 84%, 88%, or 90%. In certain embodiments the degree of deacetylation ranges from about 80% to about 90% and in certain embodiments ranges from about 82% or 84% to about 90% or 95%.

In certain embodiments the chitosans used in the nanoparticle of this invention are “high molecular weight” chitosans characterized as having a molecular weight ranging from about 500,000 to about 5,000,000 g/mole, preferably from about 800,000 to about 1,200,000 g/mole, and most preferably from about 900,000 to about 1,000,000. In certain embodiments the chitosans are lower molecular weight chitosans having a molecular weight less than about 500,000 g/mole, more typically less than about 450,000 g/mole, and still more typically less than about 400,000 g/mole. In certain embodiments the chitosans can have a Brookfield viscosity of less than 5,000 mPas; and an ash content of less than 0.3% by weight, and preferably less than 0.1% by weight, based on the weight of the chitosan.

In certain embodiments can be obtained by a process involving: (a) treating fresh crustacean shells with dilute aqueous mineral acid, (b) treating the resulting demineralized first intermediate product with aqueous alkali metal hydroxide solution, (c) treating the resulting lightly deproteinized second intermediate product with more dilute aqueous mineral acid, (d) optionally, drying the resulting decalcified third intermediate product to a residual water content of 5 to 25% by weight and (e) finally deacetylating the optionally dried product with concentrated aqueous alkali metal hydroxide, steps (a) and (c) being carried out at a temperature of about 15° C. to about 25° C. and at a pH value of 0.3 to 0.7 and steps (b) and (e) being carried out at a temperature of about 70° C. to about 110° C. and at a pH value of 12 to 14.

Suitable chitosans can also be obtained from commercial suppliers (e.g., Sigma C3646-25G (85% deacetylated)).

B) Biocompatible Polymer.

In certain embodiments the biocompatible polymer comprising the nanoparticles of the present invention is, or comprises, alginate. In certain instances, however, other biocompatible polymers can be used in conjunction with or instead of the alginate. The biocompatible polymer component can be supplied as individual biocompatible polymers or supplied in various prepared mixtures of two or more biocompatible polymers that are subsequently combined to form the biocompatible polymer component. Some illustrative, but non-limiting examples of biocompatible polymers include, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methylmethacrylate), poly(ethylmethacrylate), poly(butylmethacrylate), poly(isobutylmethacrylate), poly(hexlmethacrylate), poly(isodecylmethacrylate), poly(laurylmethacrylate), poly(phenylmethacrylate), poly(methacrylate), poly(isopropacrylate), poly(isobutacrylate), poly(octadecacrylate), polyethylene, polypropylene poly(ethylene glycol), poly(ethylene oxide), poly(ethylene terephthalate), poly(vinyl alcohols), poly(vinyl acetate), poly vinyl chloride, polystyrene, polyhyaluronic acids, casein, gelatin, gluten, polyanhydrides, polyacrylic acid, any copolymers thereof, and any combination of any of these and/or alginate.

C) Nanoparticle Formation.

The nanoparticles can be formed using a number of different protocols. In one approach, for example, 70.0 mg of alginic acid is added to 100 ml of deionized water (0.7 mg/ml) and magnetically stirred until dissolved. 15 mg of Calcium Chloride is then stirred magnetically into 20 ml of deionized water (0.75 mg/ml). The 20 ml calcium chloride (CaCl₂) solution is added dropwise into the alginic acid solution, e.g., via a 27.5 gauge needle while the alginic acid solution is concurrently being sonicated. Following administration of the CaCl₂, sonication is maintained for 1 minute. The new solution is then magnetically stirred for 0.5 hour. Following this time period, 7 mg of chitosan dissolved in 20 ml glacial acetic acid or hydrochloric acid (0.35 mg/ml) pH (3.5-4.0) is added dropwise, e.g., via a 27.5 gauge needle into the alginic acid/CaCl₂ solution. The resulting mixture in then stirred for 1 hour, and is then put aside to settle over a 24 hour time period.

After 24 hours, the now formed suspension is centrifuged, e.g., at 4000 rpm for 1 hour. The supernatant is discarded and 50 ml of deionized H₂O is put in its stead. The suspension is spun again, e.g., at 4000 rpm×1 hour. The past step is repeated twice.

Due to aggregation of the particles, the suspension can be vortexed, e.g., for two minutes, and then sonicated, e.g., for 3.5 hours.

In another approach, 70.0 mg of alginic acid is added to 100 ml of deionized H₂O (0.7 mg/ml) and stirred until dissolved. 1.5 mg of calcium chloride is then stirred into 20 ml of deionized H₂O (0.75 mg/ml). 30 mg of chitosan dissolved in 50 ml glacial acetic acid or hydrochloric acid (0.6 mg/ml) pH (3.5-4.0) is added dropwise, e.g, via a 27.5 gauge needle into the alginic acid solution. The resulting mixture in then stirred for 4 hours, and is then put aside to settle over a 24 hour time period

After 24 hours, the now formed suspension is centrifuged, e.g., at 4000 rpm for 1 hour. The supernatant is discarded and 50 ml of deionized H₂O is put in its stead. The suspension is spun again, e.g., at 4000 rpm×1 hour. The past step is repeated twice.

Due to aggregation of the particles, the suspension can be vortexed for two minutes, and then sonicated for 3.5 hours.

D) Incorporation of a Drug into the Nanoparticles(s).

Any of a wide variety of antimicrobial or other drugs can readily be incorporated into the nanoparticles. One illustrative protocol for the incorporation of benzoyl peroxide is provided below. One of skill will appreciate that similar protocols can be used with little or no modification for the incorporation of numerous other drugs.

To incorporate benzoyl peroxide, 5 g of benzoyl peroxide is dissolved into 100 ml of ethanol and spun for 1 hr. 70.0 mg alginic acid is added to the mixture and spun to dissolve. 1.5 mg of calcium chloride is then stirred into 20 ml of ethanol (0.75 mg/ml). The 20 ml calcium chloride (CaCl₂) solution is added dropwise into the alginic acid solution, e.g., via a 27.5 gauge needle while the alginic acid solution is concurrently being sonicated. Following administration of the CaCl₂, sonication is maintained for 1 minute. The resulting solution is then stirred for 0.5 hour. Following this time period, 7 mg of chitosan dissolved in 20 ml glacial acetic acid or hydrochloric acid (0.35 mg/ml) pH (3.5-4.0) is added dropwise via a 27.5 gauge needle into the alginic acid/CaCl₂ solution. The resulting mixture in then stirred for 1 hour, and is then put aside to settle over a 24 hour time period.

After 24 hours, the now formed suspension is centrifuged, e.g., at 4000 rpm for 1 hour. The supernatant is discarded and 50 ml of deionized H₂O is put in its stead. The suspension is spun again at 4000 rpm×1 hour. The past step is repeated twice. Due to aggregation of the particles, the suspension can be vortexed for two minutes, and then sonicated, e.g., for 3.5 hours.

### II. Nanoparticle Formulations.

The nanoparticles of this invention alone or when they incorporate one or more therapeutic agents can be administered alone or can be formulated as a medicament, a cosmetic, a cleanser, and the like.

The nanoparticles of this invention are particularly well suited for topical administration. Chitosan is a potent penetrant of skin and mucosa. Chitosan disrupts gap junctions and other stabilizing components of the natural barriers, thereby allowing for the particles to diffuse through freely.

While the nanoparticles and/or nanoparticles formulations of this invention are well suited for providing topical antimicrobial activity and/or topical anti-inflammatory activity, in certain embodiments, the nanoparticles can be orally, nasally, rectally, or parenterally administered.

In certain embodiments, in order to carry out the methods of the invention, one or more active agents of this invention (e.g., nanoparticles and/or nanoparticles incorporating one or more therapeutic agents) are administered, e.g. to an individual diagnosed as having acne, seborrheic dermatitis, other pathologies characterized by infection and/or inflammation or to an individual at risk for (e.g. a recurrence of) acne, seborrheic dermatitis and/or the various other pathologies described herein.

The nanoparticles can be administered alone (e.g. in a simple water or saline suspension), they can be impregnated with one or more therapeutic agents, and/or they can be administered in conjunction with one or more therapeutic agents.

The nanoparticles can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

In certain embodiments, the nanoparticles are formulated for topical administration, e.g., as an ointment, cream, gel, liquid, polymer matrix, poultice, and the like. Ointments are semisolid preparations that are typically based on petrolatum or other petroleum derivatives. Creams containing the selected active agent(s) (e.g., nanoparticles), are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

In certain embodiments, the nanoparticles of this invention are combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

In certain embodiments, the excipients are preferably sterile and generally free of undesirable matter. These compositions may be sterilized by conventional, well-known sterilization techniques.

In therapeutic applications, the compositions of this invention are administered to a patient suffering from one or more symptoms of the one or more pathologies described herein, or at risk for one or more of the pathologies described herein in an amount sufficient to prevent and/or cure and/or or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

The concentration of active agent(s) can vary widely, and will be selected primarily based on fluid volumes, viscosities, body weight and the like in accordance with the particular mode of administration selected and the patient's needs

The nanoparticles of this invention are also well suited for incorporation into various cosmetics and/or cosmetic cleansing agents. Thus, in certain embodiments, the nanoparticles are incorporated, for example, into a “facial scrub”. Formulations for various facial scrubs are well known to those of skill in the art and the nanoparticles can readily be added to various known facial scrub formulations.

Typically, a facial scrub, according to the present invention will comprise the nanoparticles of the present invention and one or more cleaning agents. Suitable cleaning agents include, but are not limited to various soaps, surfactants, and/or other amphoteric compounds. In certain embodiments, where the cleaning agents comprise one or more surfactants, the surfactant can be present in an amount ranging from about 1 to about 80%, preferably from about 5% to about 60%, more preferably from about 15% to about 45%.

In various embodiments, anionic surfactants are preferred. One suitable surfactant system is sodium cocoyl isethionate (SCI) in combination with sodium dodecyl benzene sulfonate (LAS). In certain embodiments, these materials can be advantageously used in about a 6:1 ratio (SCI:LAS). Other combinations of anionic surfactants perform acceptably. Alternate anionic materials include, but are not limited to: alkyl (C8-18) sulfates (e.g., sodium lauryl sulfate, sodium cetearyl sulfate, sodium lauryl amide methylene sulfate), alkyl (C8-18) ether sulfates (e.g., sodium laureth-x sulfate, x=1 to 12), fatty acid soaps (e.g., sodium stearate, sodium laurate), alkyl (C8-18) sulfonates (e.g., sodium C14-16 olefin sulfonate, sodium cocoglyceryl ether sulfonate, sodium lauryl sulfoacetate), sulfosuccinates (e.g., sodium lauryl sulfosuccinate, disodium laureth sulfosuccinate, isodium cocamido MEA sulfosuccinate), alkyl phosphates (sodium stearyl monophosphate, potassium lauryl phosphate), taurates (e.g., sodium methyl cocoyl taurate), alkyl (C8-18) amino acids, esters, amides and ethers thereof (e.g., acyl glutamates such as sodium cocoyl glutamate, n-lauroyl-.beta.-alanine, alkyl carboxyethylglycinates) alkyl ether carboxylates (e.g., sodium laureth-13 carboxylate) and sarcosinates (e.g., sodium lauroyl sarcosinate, sodium cocoyl sarcosinate), linear alkyl benzene sulfonates (e.g. sodium dodecyl, benzene sulfonate), and the like. In various embodiments, these materials can be neutralized with sodium, potassium, magnesium, calcium, lithium, TEA, and the like.

Nonionic surfactants such as alcohol ethers (e.g., laureth-3, steareth-6), fatty acid alkanolamides (e.g., cocamide DEA, lauramide MIPA), amine oxides (e.g., lauramine oxide, coamidopropyl amine oxide), sorbitan esters (e.g., sorbitan laurate, sorbitan oleate, sorbitan isostearate, sorbitan palmitate) and alkyl polyglucosides (e.g., decyl polyglucose, lauryl polyglucose) may be used as well, alone or in combination with anionic surfactants. These materials may be used to increase certain performance attributes such as lather or cleaning. Their inclusion will depend on the performance target for the particular product (e.g., high cleaning as opposed to skin care).

In various embodiments, zwitterionic and cationic surfactants can also be used. Acceptable surfactant classes are betaines (e.g., cocamidopropyl betaine), sultaines (e.g., cocamidopropyl hydroxysultaine), quaternary ammonium chlorides (e.g., distearyl dimethylammonium chloride, stearyl trimethyl ammonium chloride) and acylamphoacetates/acylamphopropionates (e.g., sodium lauroamphoacetate, sodium cocoamphopropionate). These surfactants are generally used for their foam building and skin feel improving properties.

The foregoing formulations and administration methods are intended to be illustrative and not limiting. It will be appreciated that, using the teaching provided herein, other suitable formulations and modes of administration can be readily devised.

III. Illustrative Drugs for Incorporation into Nanoparticles.

In various embodiments any one or more of a wide variety of antimicrobial agents and/or other pharmaceuticals can be incorporated into the nanoparticles of this invention. Such agents include, but are not limited to various antibiotics, and/or anti-inflammatory agents, steroids, non-steroidal anti-inflammatories (NSAIDs) (e.g., tacrolimus, pimecrolimus, and the like) and/or other therapeutic agents. In certain embodiments, the nanoparticles incorporate one or more therapeutic agents useful in the treatment of various skin pathologies (e.g., acne, seborrheic dermatitis, skin disease in Lupus, eczema, and the like). In certain embodiments, the nanoparticles incorporate one or more therapeutic agents selected from the group consisting of sodium bicarbonate, benzoyl peroxide, retinoids, Clindamycin, Tetracycline, flagyl, psuedomonic acid, gentimycin, Isotretinoin, Azelaic acid, Adapalene, sodium sulfacetamide, salicylic acid, sulfur, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and various steroids (e.g., corticosteroids) such as diprolene, lidex, triamcinolone, elocon, westcort, and the like.

### IV. Kits.

In another embodiment this invention provides kits for the treatment of various skin pathologies (e.g., acne, Lupus associated skin disease, eczema, psoriasis, seborrheic dermatitis, and the like), and/or infection, and/or inflammation, and the like, and/or kits for enhancing the antimicrobial and/or anti-inflammatory activity of one or one or more therapeutic agents. In various embodiments, the kits comprise a container containing one or more of the nanoparticles and/or nanoparticles impregnated with therapeutic agents as described herein. The nanoparticle(s) can be provided in a unit dosage formulation and/or may be optionally combined with one or more pharmaceutically acceptable excipients, e.g. to form a cream, ointment, gel, linament, etc.

The kit can, optionally, further comprise one or more other therapeutic agents as described herein.

In addition, the kits optionally include labeling and/or instructional materials providing directions (i.e., protocols) for the practice of the methods or use of the “therapeutics” or “prophylactics” of this invention. In certain embodiments, the instructional materials describe the use of the nanoparticles described herein in the treatment of a skin pathology (e.g. as described herein). The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Chitosan and Chitosan-Alginate Nanoparticles have Strong Antibacterial Activity against P. acnes

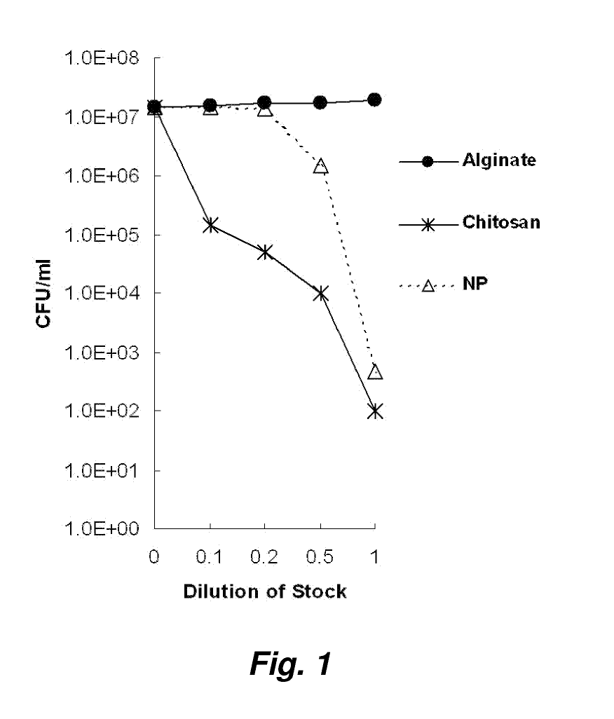

We hypothesized that both native chitosan and chitosan-alginate nanoparticles themselves have strong antibacterial action. To determine whether the chitosan and chitosan-alginate nanoparticles kill P. acnes, we tested their activity against the aforementioned microbe using the CFU assay. Both chitosan and chitosan-alginate nanoparticles exhibited antimicrobial activity in a concentration-dependent manner, reducing the number of P. acnes (see, e.g., FIG. 1).

In the P. acnes study (FIG. 1), distilled water and dissolved alginate were used as controls and as demonstrated on the graph, had no influence on the bacteria. Dissolved chitosan and (chitosan+alginate) demonstrated approximately five log of killing.

### Example 2

### Efficacy of Granulysin in Chitosan/Alginate Nanoparticles

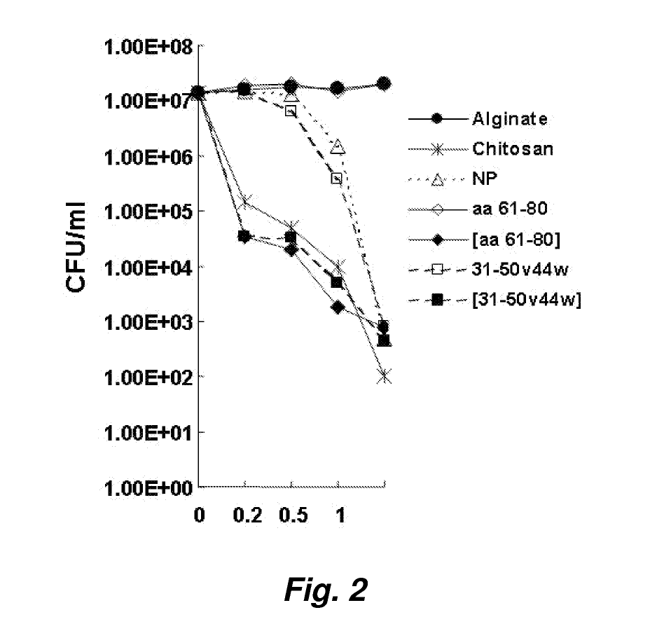

Initial studies began with the encapsulation of the antimicrobial peptide granulysin in chitosan/alginate nanoparticles. The purpose was to develop a stabile controlled release delivery vehicle for the peptide for therapeutic use against P. acnes. During CFU assay experiments, we observed that even the encapsulated non-active peptide (61-80) exerted a therapeutic impact on the bacteria while this peptide alone did not (see, e.g., FIG. 2).

### Chitosan and Chitosan-Alginate Nanoparticles have Strong Antibacterial Activity against S. aureus and E. coli

The activity of chitosan and chitosan-alginate nanoparticles against the pathogens S. aureus and E. coli was also studied.

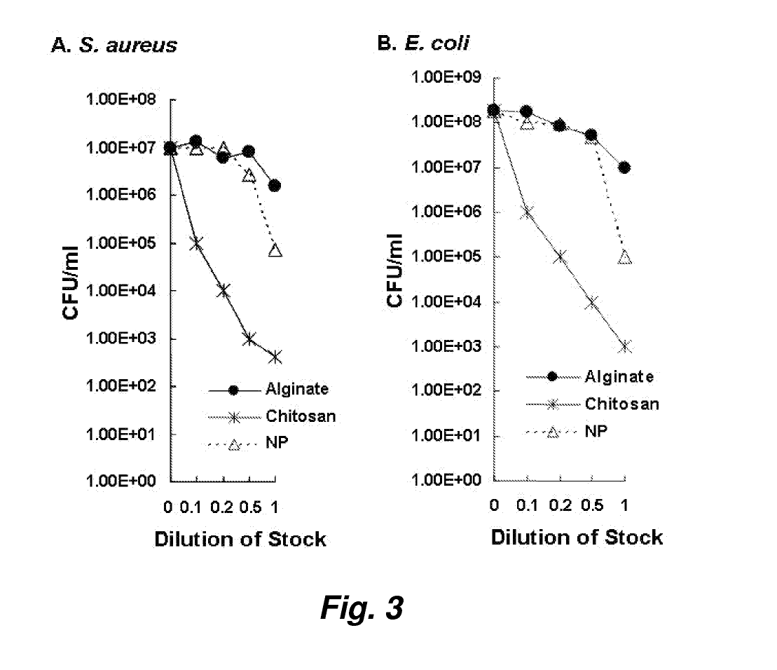

The S. aureus experiments (FIG. 3, panel A) illustrate the progressive increase in killing from native chitosan (1 and 2 logs of killing respectively), to chitosan plus alginate (2-3 logs of killing respectively), to encapsulated chitosan nanoparticles dispersed in 5 ml of distilled water at pH 3.0 (2-3.5 logs of killing). Like the P. acnes study, the distilled water and alginate had no significant impact on the bacteria.

The E. coli experiments (FIG. 3, panel A, panel B) demonstrate chitosan's ability to retard bacterial growth. Both dissolved chitosan and dissolved (chitosan plus alginate) illustrated a greater killing impact of approximately 4 logs each, then the nanoparticles, which demonstrated 2.5 logs. Without being bound to a particular theory, it is believed that the acidity of the suspension may play a role in the therapeutic index of the particles.

### Evaluation of Chitosan-Alginage Nanoparticles with Other Drugs

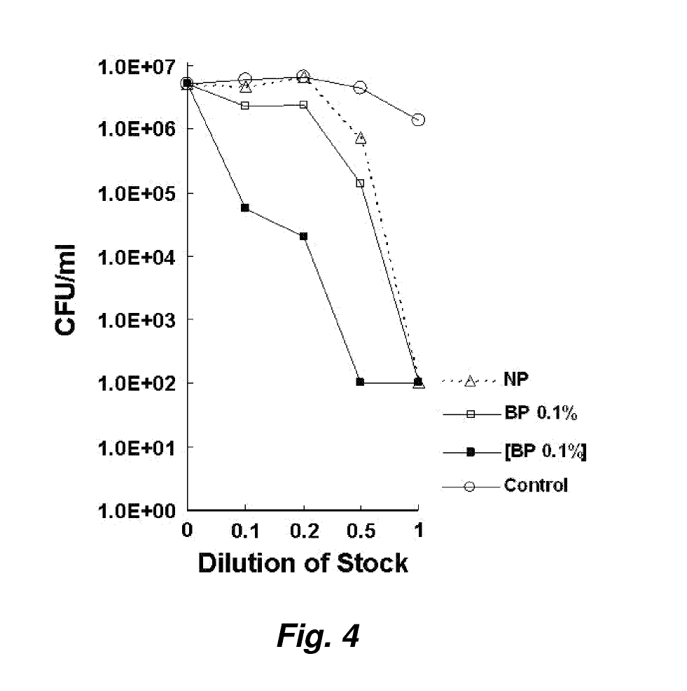

Encapsulation of benzoyl peroxide with nanoparticle enhanced the killing of P. acnes (see, e.g., FIG. 4).

As mentioned earlier, chitosan's properties are influenced by environmental pH.

### Chitosan-Alginage Nanoparticles have Anti-inflammatory Properties

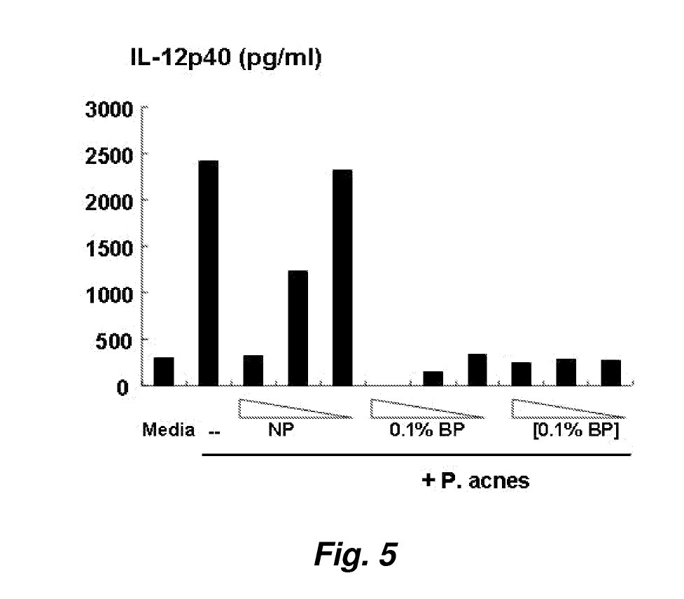

Because inflammation is known to be a key pathogenic factor in Acne vulgaris, we evaluated the impact of the nanoparticles on the production of IL-12p40 from human cells exposed to P. acnes. FIG. 5 demonstrates that nanoparticles can decrease the production of IL-12p40 and the extent to which this inhibition occurs is therapeutic dose-dependent

### Methods

Chitosan>85% deacetylated (C3646-25G) and Alginic Acid sodium salt (A2158-100G) were both acquired from Sigma. Calcium chloride dihydrate Reagent ACS (lot: A0200178001) was purchased from Acros Organics. Glacial Acetic Acid (A490-212) and Hydrochloric Acid (A144-500) were acquired from Fisher Scientific.

Protocol 1:

70.0 mg of alginic acid was added to 100 ml of deionized H₂O (0.7 mg/ml) and magnetically stirred until dissolved. 15 mg of calcium chloride was then stirred magnetically into 20 ml of deionized H₂O (0.75 mg/ml). The 20 ml calcium chloride (CaCl₂) solution was added dropwise into the alginic acid solution via a 27.5 gauge needle while the alginic acid solution was concurrently being sonicated. Following administration of the CaCl₂, sonication was maintained for 1 minute. The new solution was then magnetically stirred for 0.5 hour. Following this time period, 7 mg of chitosan dissolved in 20 ml glacial acetic acid or hydrochloric acid (0.35 mg/ml) pH (3.5-4.0) is added dropwise via a 27.5 gauge needle into the alginic acid—CaCl₂ solution. The resulting mixture was then stirred magnetically for 1 hour, and then put aside to settle over a 24 hour time period.

After 24 hours, the formed suspension was centrifuged at 4000 rpm for 1 hour. The supernatant is discarded and 50 ml of deionized H₂O is put in its stead. The suspension is spun again at 4000 rpm×1 hour. The past step was repeated twice. Due to aggregation of the particles, the suspension was vortexed for two minutes, and then sonicated×3.5 hours.

Protocol 2:

70.0 mg of alginic acid was added to 100 ml of di. H₂O (0.7 mg/ml) and magnetically stirred until dissolved. 1.5 mg of calcium chloride was then stirred magnetically into 20 ml of deionized H₂O (0.75 mg/ml). 30 mg of chitosan dissolved in 50 ml glacial acetic acid or hydrochloric acid (0.6 mg/ml) pH (3.5-4.0) was added dropwise via a 27.5 gauge needle into the alginic acid solution. The resulting mixture was then stirred magnetically for 4 hours, and then put aside to settle over a 24 hour time period.

After 24 hours, the suspension was centrifuged at 4000 rpm for 1 hour. The supernatant was discarded and 50 ml of deionized H₂O was put in its stead. The suspension was spun again at 4000 rpm×1 hour. The past step was repeated twice. Due to aggregation of the particles, the suspension was vortexed for two minutes, and then sonicated×3.5 hours.

Protocol 3:

5 g of benzoyl peroxide was dissolved into 100 ml of ethanol and spun for 1 hr. 70.0 mg alginic acid was added to the mixture and spun to dissolve. 1.5 mg of calcium chloride was then stirred magnetically into 20 ml of ethanol (0.75 mg/ml). The 20 ml calcium chloride (CaCl₂) solution was added dropwise into the alginic acid solution via a 27.5 gauge needle while the alginic acid solution was concurrently being sonicated. Following administration of the CaCl₂, the sonication was maintained for 1 minute. The new solution was then magnetically stirred for 0.5 hour. Following this time period, 7 mg of chitosan dissolved in 20 ml glacial acetic acid or hydrochloric acid (0.35 mg/ml) pH (3.5-4.0) was added dropwise via a 27.5 gauge needle into the alginic acid/CaCl₂ solution. The resulting mixture was then stirred magnetically for 1 hour, and was then put aside to settle over a 24 hour time period

After 24 hours, the formed suspension was centrifuged at 4000 rpm for 1 hour. The supernatant was discarded and 50 ml of deionized H2O is put in its stead. The suspension was spun again at 4000 rpm×1 hour. The past step was repeated twice. Due to aggregation of the particles, the suspension was vortexed for two minutes, and then sonicated×3.5 hours.

### Discussion

We have demonstrated that chitosan nanoparticles have strong antibacterial properties against P. acnes, S. aureus, and E. coli. The nanoparticles of chitosan not only provide a delivery system for therapeutic compounds, but also allow for delayed and continued release of this antimicrobial property. The extents to which these nanoparticles can be applied seem limitless. And moreover, because these antimicrobial agents can be combined with other therapeutics, offering both its curative character and its confirmed delayed release, these chitosan nanoparticles allow us to advance and improve upon our current medicinal battery.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An antimicrobial composition, said composition comprising:
particles comprising chitosan and alginate where said particles have antimicrobial activity.

2. The antimicrobial composition of claim 1, wherein said particles are nanoparticles.

3. The antimicrobial composition of claim 1, wherein said particles are nanoparticles having an average diameter of less than about 500 nm.

4-5. (canceled)

6. The antimicrobial composition of claim 1, wherein said particles have antimicrobial activity against bacteria selected from the group consisting of S. aureus, B. circulans, B. cereus, E. coli, P. vulgaris, P. acnes, S. enterica, V. anguillarum, K. pneumoniae, P. piscicida, P. aeruginosa, A. tumefaciens, C. micgiganence, A. mali, E. chrysanthemi, X. campestris, C. diplodiella, and P. piricola.

7. The antimicrobial composition of claim 1, wherein said chitosan is partially or fully deacetylated.

8. The antimicrobial composition of claim 7, wherein said chitosan is at least 50% deacetylated.

9-10. (canceled)

11. The antimicrobial composition of claim 1, wherein said particles are impregnated with a biologically active agent.

12. The antimicrobial composition of claim 11, wherein said biologically active agent is an agent for the treatment of a skin disorder.

13. The antimicrobial composition of claim 11, wherein said biologically active agent is selected from the group consisting of sodium bicarbonate, benzoyl peroxide, retinoids, retin A, Clindamycin, Tetracycline, flagyl, psuedomonic acid, gentimycin, Isotretinoin, Azelaic acid, Adapalene, sodium sulfacetamide, salicylic acid, sulfur, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and steroids.

14. The antimicrobial composition of claim 11, wherein said biologically active agent is selected from the group consisting of diprolene, lidex, triamcinolone, elocon, and westcort.

15. A method of mitigating one or more symptoms of a skin pathology characterized by a microbial infection and/or inflammation, said method comprising:
contacting the infected skin with a composition comprising particles comprising chitosan and alginate where said particles have antimicrobial activity.

16. The method of claim 15, wherein said particles are nanoparticles.

17. The method of claim 15, wherein said particles are nanoparticles having an average diameter of less than about 500 nm.

18-19. (canceled)

20. The method of claim 15, wherein said particles have antimicrobial activity against bacteria selected from the group consisting of S. aureus, B. circulans, B. cereus, E. coli, P. vulgaris, P. acnes, S. enterica, V. anguillarum, K. pneumoniae, P. piscicida, P. aeruginosa, A. tumefaciens, C. micgiganence, A. mali, E. chrysanthemi, X. campestris, C. diplodiella, and P. piricola.

21. The method of claim 15, wherein said chitosan is partially or fully deacetylated.

22-24. (canceled)

25. The method of claim 15, wherein said particles are impregnated with a biologically active agent.

26. The method of claim 25, wherein said biologically active agent is an agent for the treatment of a skin disorder.

27. The method of claim 25, wherein said biologically active agent is selected from the group consisting of benzoyl peroxide, retin A, Clindamycin, Tetracycline, Isotretinoin, Azelaic acid, Adapalene, sodium Sulfacetamide, salicylic acid, sulfur, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and steroids.

28. A method of reducing the inflammation of a tissue, said method comprising contacting said tissue with a composition comprising particles comprising chitosan and alginate where said particles have anti-inflammatory activity.

29. The method of claim 28, wherein said particles are nanoparticles.

30-32. (canceled)

33. The method of claim 28, wherein said inflammation is inflammation associated with acne or seborrheic dermatitis.

34. The method of claim 28, wherein said chitosan is partially or fully deacetylated.

35-37. (canceled)

38. The method of claim 28, wherein said particles are impregnated with a biologically active agent.

39. The method of claim 38, wherein said biologically active agent is an agent for the treatment of a skin disorder.

40. The method of claim 38, wherein said biologically active agent is a steroid or other anti-inflammatory agent.

41. A method of increasing the antimicrobial and/or anti-inflammatory activity of a biologically active agent, said method comprising combining said agent with particles comprising chitosan and alginate where said particles have antimicrobial activity.

42. The method of claim 41, wherein said particles are nanoparticles.

43-45. (canceled)

46. The method of claim 41, wherein said particles have antimicrobial activity against bacteria selected from the group consisting of S. aureus, B. circulans, B. cereus, E. coli, P. vulgaris, P. acnes, S. enterica, V. anguillarum, K. pneumoniae, P. piscicida, P. aeruginosa, A. tumefaciens, C. micgiganence, A. mali, E. chrysanthemi, X. campestris, C. diplodiella, and P. piricola.

47. The method of claim 41, wherein said chitosan is partially or fully deacetylated.

48-50. (canceled)

51. The method of claim 41, wherein said biologically active agent is an agent for the treatment of a skin disorder.

52. The method of claim 51, wherein said biologically active agent is selected from the group consisting of benzoyl peroxide, retin A, Clindamycin, Tetracycline, Isotretinoin, Azelaic acid, Adapalene, sodium Sulfacetamide, salicylic acid, sulfur, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and steroids.

53. A facial scrub, said facial scrub comprising:
a cleaning agent; and
antimicrobial particles comprising chitosan and alginate.

54. The facial scrub of claim 53, wherein said cleaning agent comprises a surfactant.

55. The facial scrub of claim 53, wherein said cleaning agent comprises an anionic surfactant.

56. The facial scrub of claim 53, wherein said scrub further comprises an abrasive particle.

57. The facial scrub of claim 53, wherein said antimicrobial particles are nanoparticles.

58-60. (canceled)

61. The facial scrub of claim 53, wherein said antimicrobial particles have antimicrobial activity against bacteria selected from the group consisting of S. aureus, B. circulans, B. cereus, E. coli, P. vulgaris, P. acnes, S. enterica, V. anguillarum, K. pneumoniae, P. piscicida, P. aeruginosa, A. tumefaciens, C. micgiganence, A. mali, E. chrysanthemi, X. campestris, C. diplodiella, and P. piricola.

62. The facial scrub of claim 53, wherein said chitosan is partially or fully deacetylated.

63-65. (canceled)

66. The facial scrub of claim 53, wherein said antimcriobial particles are impregnated with a biologically active agent.

67. The facial scrub of claim 66, wherein said biologically active agent is an agent for the treatment of a skin disorder.

68. The facial scrub of claim 66, wherein said biologically active agent is selected from the group consisting of benzoyl peroxide, retin A, topical Clindamycin, topical Tetracycline, topical Isotretinoin, Azelaic acid, Adapalene, sodium Sulfacetamide, salicylic acid, sulfur, resorcinol, vitamin A, vitamin D, resorcinol, vitamin A, vitamin D, vitamin C, vitamin E, vitamin K, vitamin B, Elidel, antipruritics, hyaluronic acid, collagen, botox, anti-oxidants including genestein, ubiquinone, polypheols, and steroids.

69. An anti-microbial composition, said composition comprising:
particles comprising chitosan and a hydrophilic biocompatible polymer, wherein said particles have antimicrobial activity.

70. The antimicrobial composition of claim 69, wherein said biocompatible polymer is selected from the group consisting of polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methylmethacrylate), poly(ethylmethacrylate), poly(butylmethacrylate), poly(isobutylmethacrylate), poly(hexlmethacrylate), poly(isodecylmethacrylate), poly(laurylmethacrylate), poly(phenylmethacrylate), poly(methacrylate), poly(isopropacrylate), poly(isobutacrylate), poly(octadecacrylate), polyethylene, polypropylene poly(ethylene glycol), poly(ethylene oxide), poly(ethylene terephthalate), poly(vinyl alcohols), poly(vinyl acetate), poly vinyl chloride, polystyrene, polyhyaluronic acids, casein, gelatin, gluten, polyanhydrides, polyacrylic acid, alginate, any copolymers thereof, and any combination of any of these.
